(19) United States
(12) Patent Application Publication
(10) Pub. No.: US 2023/0095133 A1
(43) Pub. Date: Mar. 30, 2023

(54) EXTRAORAL VACUUM AEROSOL CUP AND METHOD OF USE

(71) Applicants: Peter Liu, Ambler, PA (US); Hai Qing, New Britain, PA (US)

(72) Inventors: Peter Liu, Ambler, PA (US); Hai Qing, New Britain, PA (US)

(21) Appl. No.: 17/951,966

(22) Filed: Sep. 23, 2022

Related U.S. Application Data
(60) Provisional application No. 63/248,144, filed on Sep. 24, 2021.

(51) Int. Cl. A61C 17/08

(52) U.S. Cl. CPC A61C 17/08

(57) ABSTRACT

An affordable, reusable or one-time usage cup which enables effective and efficient collecting and removing of ejected aerosols/droplets from the mouths and/or nasal passages of dental patients but with enough room to still perform any dental procedure. The cup is connected to a vacuum source. The design of the cup is generally funnel-like and includes a cover which catches large debris ejected from the dental patient. The cup may include a variable height rim surrounding the input opening and significantly removes any aerosols/debris ejected from the patient thereby lowering the exposure of the dental professional to airborne and surface contaminants. The cup cover, occluding the main cup opening, includes holes through which any aerosols/droplets pass.

### CROSS-REFERENCE TO RELATED PATENT APPLICATIONS

This patent application claims priority to and benefit of U.S. Provisional Patent Application No. 63/248,144 filed Sep. 24, 2021, which is incorporated by reference herein for all purposes.

### TECHNICAL FIELD

This disclosure relates to a device and method of using the device to capture aerosols exiting from a patient's mouth during oral procedures (e.g., dental examination, cleaning, surgery, other procedures, etc.).

### BACKGROUND

Dental patients and healthcare providers (e.g., dental professionals such as dentist, hygienist, oral surgeons, etc.) can be exposed to a variety of pathogens including viruses, bacteria, chemical odor, and dust, due to frequent exposure to saliva, blood, and other body fluids (principally from the mouth and surrounding areas of their patients) within the dental care environment. It is well reported that in dental clinics, hospitals, or any facility where oral procedures may be performed, droplet and/or aerosol transmission of infectious diseases constitute a major concern, due to the generation of large amounts of aerosol and droplets mixed with patient's saliva during a potentially infected patient's breathing (or cough/sneeze), as well as the generation of aerosol and droplets resulting from the operation of dental devices such as high-speed dental hand-piece (e.g., drill, brush, polisher, etc.) with running water or gas. These expelled aerosols and droplets include the possibility of, for example, the spread of the SARS-COVID virus. And these droplets/aerosols are small enough to stay airborne for an extended period before they settle on environmental surfaces or enter the respiratory tract of exposed individuals. Thus, there is a high risk of spreading viruses, bacteria, chemicals, or dust, through droplets and aerosols from infected individuals in dental clinics and hospitals. Presently, any type of extraoral suction device/unit is rarely being used during these procedures. Further, the existing dental saliva ejector is designed for suction of saliva spit, and is not suitable for the suction of aerosols/fine droplets.

While there are extraoral suction devices on the market, the existing devices have high cost (>$2,000) and are rarely used in the dental office. Some major issues with these units are the noise produced by the vacuum machine and their bulky size, which is not suitable as an add on for typical compact dental rooms (or other locations where oral procedures are performed). Due to the need for effective and low cost aerosol/droplet capture, some dentists resort to DIY suction cups made from regular paper cups, but these DIY cups have poor durability and poor aerodynamic suction performance. What is needed is an affordable, 3D printable, and aerodynamically optimized Aerosol Evacuation Cup that can be directly connected to an existing central vacuum standard high-volume evacuator (HVE) valve in a typical dental office.

### SUMMARY

This disclosure describes a novel affordable aerodynamically optimized aerosol excavation cup, described as an extraoral (meaning outside the mouth) vacuum aerosol cup (or EVAC for short) that can be directly connected to existing standard central vacuum high-volume evacuator (HVE) systems in a typical dental office. The EVAC is a smaller, less expensive to manufacture, and lighter addition to an already existing vacuum system, presenting an innovative way of accomplishing and optimizing aerosol excavation. The evacuation cup can be both sterilizable and reusable, or disposable. The dimensions and geometry of the oral evacuation device are designed according to human physiological geometry for efficient aerosol suction while leaving a comfortable amount of room for dental procedures/operations. Computational fluid dynamics (CFD) simulation was performed to optimize the designed cup shape with consideration of suction vacuum pressure provided by central vacuum connected to HVE. A few variations in design are proposed such as the angled cup for dental assistants to more easily hold it and the cup design for coverage of the human mouth, both of which provide for extra dental operation space. The device can be manufactured from a wide range of materials.

At least some of the above-noted technological benefits can be obtained from a device including a hollow funnel (a conical shape with a wider and a narrower opening at the two ends, for example, a cone) shaped main body, wherein the main body has a first opening at a first end of the main body and a second opening at a second end of the main body; a rim located at the first end of the main body and surrounding the first opening; and a coupler portion located at the second end of the main body.

The device may further comprise a cover sized to occlude the first opening, wherein the cover includes a plurality of holes. The rim of the device may have a rim height which may not be uniform around the entire first opening. In some embodiments, the rim height is constant for one half of the first opening and variable for the other half of the first opening. The variable rim height may be larger than the constant rim height. In some embodiments, the rim height is constant for a first portion of the first opening and variable for a second portion of the first opening.

In some embodiments, the plurality of holes in the cover of the device are all of equal size and are equally distributed over the cover. In some embodiments, the plurality of holes are distributed more towards the side of the main body with a higher rim height. In some embodiments, the holes are not all of equal size.

The coupler portion of the device may be sized to snuggly fit into or around a vacuum attachment. In other embodiments, the coupler portion is elongated and includes a bend to facilitate placement of the device near a patient's head.

In an embodiment, a method comprises connecting the device described above to an external vacuum source; placing the device near a patient's mouth during a dental procedure; modifying the suction strength to achieve expected results.

### DESCRIPTION OF DRAWINGS

FIG. 1 illustrates a computer aided design of the Extraoral Vacuum Aerosol Cup (EVAC);

FIGS. 2A and 2B illustrates a contour design of the EVAC relative to a patient's head/face and how the EVAC would be connected to an existing high-volume evacuator (HVE) as well as illustrating the capture of expelled aerosols;

FIG. 3 illustrates a 3-dimensional representation of an embodiment of a EVAC device;

FIG. 4 illustrates a side view of the device shown in FIG. 3;

FIG. 5 illustrates a top view of the device shown in FIG. 3 (but without a cover as described herein);

FIG. 6 illustrates a 3-dimensional diagram of another embodiment of a EVAC device;

FIG. 7A is a perspective view of one embodiment of the EVAC device

FIG. 7B illustrates a top view of the device shown in FIG. 7A; and

FIG. 8 illustrates computer aerosol modeling on a simulated patient.

### DETAILED DESCRIPTION

Generally, this disclosure enables effective and efficient collecting and removing of ejected aerosols/droplets from the mouths and/or nasal passages of dental patients. For example, this disclosure describes an affordable, reusable or one-time usage cup, specifically designed for this purpose that can be placed near the mouth of the dental patient but with enough room to still perform any dental procedure, the cup being configured to connect to a vacuum source. The design of the cup catches large debris ejected from the dental patient and significantly removes any aerosols/debris ejected from the patient thereby lowering the exposure of the dental professional to airborne and surface contaminants.

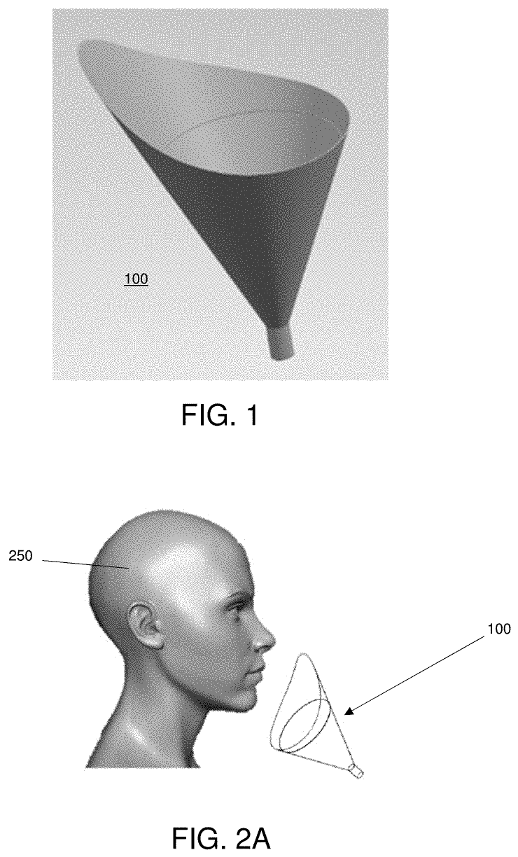

This disclosure is now described more fully with reference to FIGS. 1-8, in which some embodiments of this disclosure are shown. This disclosure may, however, be embodied in many different forms and should not be construed as necessarily being limited to the embodiments disclosed herein. Rather, these embodiments are provided so that this disclosure is thorough and complete, and fully conveys various concepts of this disclosure to skilled artisans.

Various terminology used herein can imply direct or indirect, full or partial, temporary or permanent, action or inaction. For example, when an element is referred to as being “on,” “connected,” or “coupled” to another element, then the element can be directly on, connected, or coupled to another element or intervening elements can be present, including indirect or direct variants. In contrast, when an element is referred to as being “directly connected” or “directly coupled” to another element, then there are no intervening elements present.

As used herein, various singular forms “a,” “an,” and “the” are intended to include various plural forms (e.g., two, three, four, five, six, seven, eight, nine, ten, tens, hundreds, thousands) as well, unless specific context clearly indicates otherwise.

As used herein, various presence verbs “comprises,” “includes” or “comprising,” “including” when used in this specification, specify a presence of stated features, integers, steps, operations, elements, or components, but do not preclude the presence or addition of one or more other features, integers, steps, operations, elements, components, or groups thereof.

As used herein, a term “or” is intended to mean an inclusive “or” rather than an exclusive “or.” That is, unless specified otherwise, or clear from context, “X employs A or B” is intended to mean any of a set of natural inclusive permutations. That is, if X employs A; X employs B; or X employs both A and B, then “X employs A or B” is satisfied under any of the foregoing instances.

As used herein, a term “or others,” “combination”, “combinatory,” or “combinations thereof” refers to all permutations and combinations of listed items preceding that term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. Skilled artisans understand that typically there is no limit on number of items or terms in any combination, unless otherwise apparent from the context.

As used herein, unless otherwise defined, all terms (including technical and scientific terms) used herein have the same meaning as commonly understood by one of ordinary skill in an art to which this disclosure belongs. Various terms, such as those defined in commonly used dictionaries, should be interpreted as having a meaning that is consistent with a meaning in a context of a relevant art and should not be interpreted in an idealized or overly formal sense unless expressly so defined herein.

As used herein, relative terms such as “below,” “lower,” “above,” and “upper” can be used herein to describe one element's relationship to another element as illustrated in the set of accompanying illustrative drawings. Such relative terms are intended to encompass different orientations of illustrated technologies in addition to an orientation depicted in the set of accompanying illustrative drawings. For example, if a device in the set of accompanying illustrative drawings were turned over, then various elements described as being on a “lower” side of other elements would then be oriented on “upper” sides of other elements. Similarly, if a device in one of illustrative figures were turned over, then various elements described as “below” or “beneath” other elements would then be oriented “above” other elements. Therefore, various example terms “below” and “lower” can encompass both an orientation of above and below.

As used herein, a term “about” or “substantially” refers to a +/−10% variation from a nominal value/term. Such variation is always included in any given value/term provided herein, whether or not such variation is specifically referred thereto.

Features described with respect to certain embodiments may be combined in or with various some embodiments in any permutational or combinatory manner. Different aspects or elements of example embodiments, as disclosed herein, may be combined in a similar manner.

Although the terms first, second, third, etc. can be used herein to describe various elements, components, regions, layers, or sections, these elements, components, regions, layers, or sections should not necessarily be limited by such terms. These terms are used to distinguish one element, component, region, layer or section from another element, component, region, layer or section. Thus, a first element, component, region, layer, or section discussed below could be termed a second element, component, region, layer, or section without departing from various teachings of this disclosure.

Features described with respect to certain example embodiments can be combined and sub-combined in or with various other example embodiments. Also, different aspects or elements of example embodiments, as disclosed herein, can be combined and sub-combined in a similar manner as well. Further, some example embodiments, whether individually or collectively, can be components of a larger system, wherein other procedures can take precedence over or otherwise modify their application. Additionally, a number of steps can be required before, after, or concurrently with example embodiments, as disclosed herein. Note that any or all methods or processes, at least as disclosed herein, can be at least partially performed via at least one entity in any manner.

Example embodiments of this disclosure are described herein with reference to illustrations of idealized embodiments (and intermediate structures) of this disclosure. As such, variations from various illustrated shapes as a result, for example, of manufacturing techniques or tolerances, are to be expected. Thus, various example embodiments of this disclosure should not be construed as necessarily limited to various particular shapes of regions illustrated herein, but are to include deviations in shapes that result, for example, from manufacturing.

Any or all elements, as disclosed herein, can be formed from a same, structurally continuous piece, such as being unitary, or be separately manufactured or connected, such as being an assembly or modules. Any or all elements, as disclosed herein, can be manufactured via any manufacturing processes, whether additive manufacturing, subtractive manufacturing, or any other types of manufacturing. For example, some manufacturing processes include three dimensional (3D) printing, laser cutting, computer numerical control routing, milling, pressing, stamping, vacuum forming, hydroforming, injection molding, lithography, and so forth.

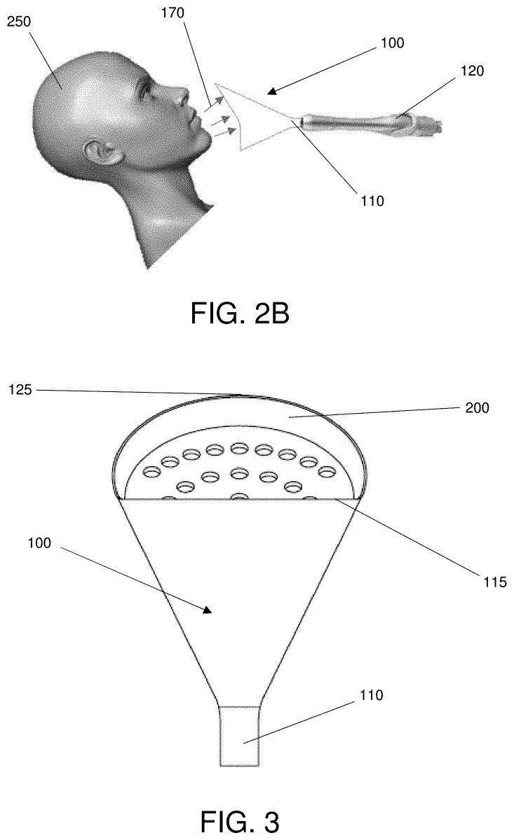

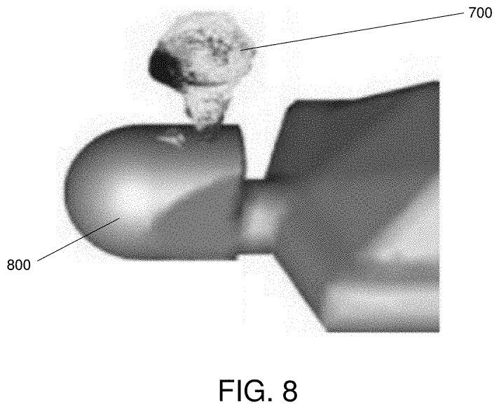

FIG. 1 shows a CAD design of the Extraoral Vacuum Aerosol Cup. The cup 100 is generally funnel shaped; the cup 100 may also be described as “cone-like.” FIG. 2A illustrates that the shape of the Extraoral Vacuum Aerosol Cup fits closely with human face anatomically. And FIG. 8 shows a computational fluid dynamics simulation to optimize the aerodynamics performance of the cup 100.

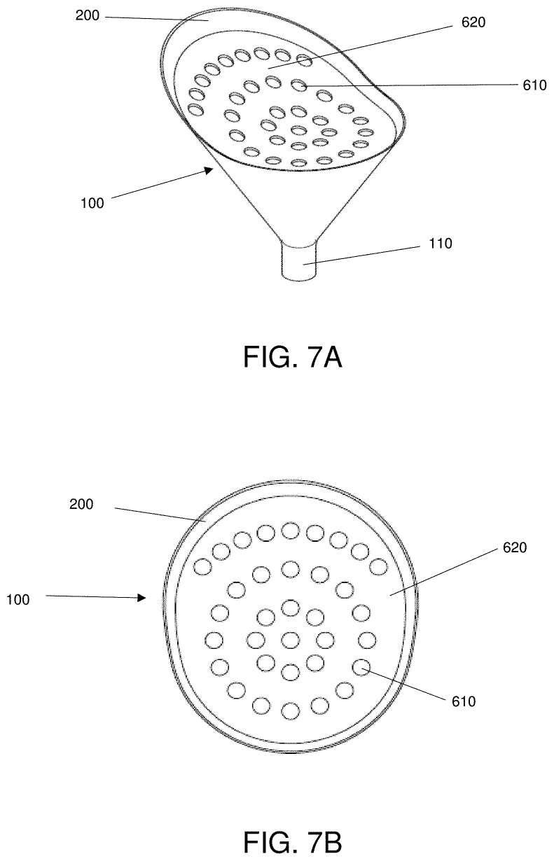

As shown in FIGS. 1 and 7A, the cup 100 includes a rim 200 with multiple heights; that is the design dimensions and geometry of the oral evacuation cup are contoured according to the anatomy of human lower face for efficient aerosol suction while leaving comfortable room for operations of dental professionals. FIG. 2A illustrates the contoured rim in relation to the patient's 250 head. The rim 200 of the cup 100 extends outwards (i.e., higher than other portions of the rim) to increase surface area and capture stray particles. FIG. 2B shows how the contoured rim 200 more efficiently captures aerosols 170. Computational fluid dynamics simulation were performed to optimize the designed cup shape with consideration of suction vacuum pressure and volume flow rate provided by central vacuum connected to HVE. An exemplary fluid dynamics simulation is shown in FIG. 8 and shows a modeled aerosol cloud 700 of a simulated patient 800 to demonstrate the risk of contamination from aerosols/droplets. The simulation shows that after a certain distance away from the contaminate source (i.e., patient mouth/nose) a larger surface area of the cup opening 190 is needed.

The design with different rim 200 heights creates an increased surface area for air flow/suction without an increase in the general diameter of the cup 100 opening 190. Further the different heights were chosen to follow a natural contour of a patient's 250 mouth/face area. This allows the cup 100 to be held in a more natural position slightly more parallel to the patient's position. In embodiments without the contoured rim (i.e., with a rim of the same height all the way around the cup opening 190), the cup 100 would have to be held in more of a 90° orientation (that is straight in front of patient's mouth) to achieve the same suction profile.

FIGS. 3, 4, 5, 6, 7A, and 7B, all show the contoured rim feature. It should be noted that the contoured rim is not necessary but does provide additional benefits. In some embodiments, the cup 100 is skewed upwards at an angle to maximize particle (aerosols/droplets) capture, while maintaining a clear view of the mouth so the dental practitioner can perform procedures without distraction.

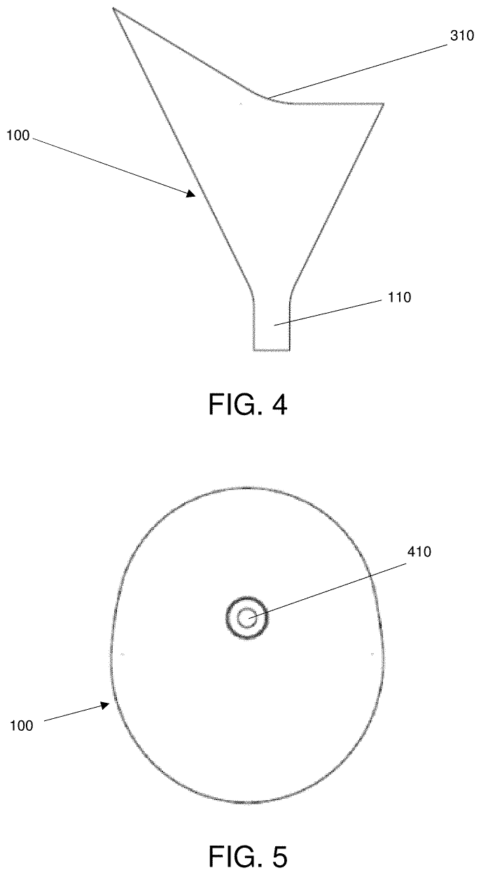

FIG. 3 is a 3-dimensional illustration showing one height 115 of the rim 200 and a second height 125 which are different. The rim 200 portions connecting these two heights can vary in an number of ways: linearly, geometrically, exponentially, stepped, or any other mathematical relationship. FIG. 4 shows an inflection point 310 where the rim height changes from a constant height on the right (from the perspective shown in FIG. 4) of inflection point 310 to a variable height around the rim up to the peak height 125 on the left (from the perspective shown in FIG. 4) of inflection point 310. In this example, the rim 200 height changes linearly. The curved rim 200 shape allows for increased operation room for dental professionals to work while maintaining coverage for capturing aerosols/droplets. As noted above, the variable height rim creates an increased surface area for air flow/suction without an increase in the general diameter of the cup 100 opening 190. In an exemplary embodiment, the one rim height 115 may be between 0%-33% of the maximum outer diameter of the cup 100. In another exemplary embodiment, the one rim height 115 may be between 5%-20% of the maximum outer diameter of the cup 100. In other exemplary embodiments, the one rim height 115 may be 0, 1, 2, 3, 4, 5, 6, 7, 8, 9, 10, 11, 12, 13, 14, 15, 16, 17, 18, 19, or 20% of the maximum outer diameter of the cup 100. In an exemplary embodiment, the second rim height 125 may be between 0%-80% of the maximum outer diameter of the cup 100. In another exemplary embodiment, the second rim height 125 may be between 10%-66% of the maximum outer diameter of the cup 100. In other exemplary embodiments, the second rim height 125 may be 0%-5%, 5%-10%, 10%-15%, 15%-20%, 20%-25%, 25%-30%, 30%-35%, 35%-40%, 40%-45%, 45%-50%, or 50%-66% of the maximum outer diameter of the cup 100.

FIG. 5 show how the different heights of the rim 200 create an increased surface area of the cup opening 190 to improve the suction coverage of the cup 100. An opening 410 located within the coupler portion 110 is where an existing vacuum extractor is coupled to the cup 100. The opening 410 is hollowed for air communication between inside of cup and vacuum.

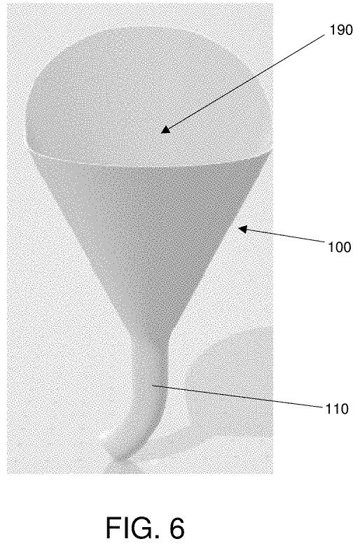

The contoured rim design is clearly seen in the 3-dimensional illustration shown in FIG. 6. This figure illustrates an embodiment without the cover 620. However, the cover 620 can likewise be attached to the cup 100.

As shown in FIG. 2A, the contoured rim 200 is such that the extended (larger height rim) can be located in line with or near the nasal area of the patient 250. This area appears to receive more aerosol/droplets when a patient is positioned prone or semi-prone. As shown in FIG. 2B, aerosols/droplets 170 may be in higher concentrations near the top of the evacuator cup due to the prone-like nature of the patient's 250 oral and nasal cavity during procedures.

Additionally, designs such as the angled cup 100 for easy holding by dental professionals are contemplated. FIG. 2B illustrates the usage of the device, where the device could be either held by the patient 250 close to the mouth (the distance to mouth can be freely adjusted), held by a separate fixer equipment, or held by a dental professional (a variation design is shown in FIG. 6). In FIG. 6, the coupler portion 110 which is configured for coupling or mating to an HVE already present in the procedure room, is further elongated and optionally curved or bent. Coupler portion 110 is located at the bottom of the cup 100 and fits to conventional dental vacuum system. FIG. 2A shows the coupler portion 110 mated (or coupled to) with a vacuum attachment 120 of the HVE unit. The variable features of the coupler 110 facilitate grip and positioning by either the patient 250, office personal, or separate fixer equipment. The cup 100 can be held at a reasonable position in front of a patient by hand or mechanical clamp.

The outer diameter of the cup 100 may be from 2 cm to 20 cm. The outer diameter may be variable when the cup 100 is shaped like a funnel or cone; in such embodiments, the 2 cm to 20 cm diameter may represent the minimum (smaller end of funnel/cone) and maximum (larger end of funnel/cone) variable outer diameter. The thickness of the cup wall may be from 0.1 mm to 5 mm. The outer diameter of the connection tube 110 may be from 1 cm to 5 cm. The length of the connection tube 110 may be from 0.1 cm to 50 cm. The shape of the smart extraoral vacuum aerosol cup can be smooth curve or straight cone or a combination thereof. The above-noted measurements of the outer diameter of the cup 100, the thickness of the cup wall, the outer diameter of the connection tube 110, and the length of the connection tube 110 are not exhaustive. Values of these measurements which may decrease or increase with relative scaling of the entire cup 100 have been contemplated.

The smart extraoral vacuum aerosol cup 100 may include an open cup outlet 190, the outlet 190 may also be covered with the perforated surface (i.e., cover) 620 having holes 610 to optimize the air dynamics, as shown in FIGS. 7A and 7B. The cover 620 may be coupled at the first opening to the main body in a plane perpendicular to the main body central axis or may be coupled to the main body in a plane angled to match or similarly follow the shape of the differing rim heights. This cover 620 with holes 610 structure accomplishes at least two features: first, it concentrates the suction power of the larger cup into the smaller holes lowering the surface area of the suction opening and thus increase the localized power and efficiency of the suction; and second, the cover 620 functions to capture larger debris which is preferably not suctioned into the vacuum anyway. The holes 610 on the cover 620 of the cup are the only exposure sites and thus increase vacuum pressure. A substantially solid cover 620 prevents larger objects from clogging the vacuum. The holes 610 may be uniformly positioned around the cover 620. Alternatively, the holes 610 may be distributed more heavily towards the skewed side of the cup 100. In one embodiment, the size (e.g., diameter) of each of the holes 610 does not have to be uniform among a plurality of holes 610. As noted above, the holes are sized to concentrate the suction power of the larger cup while capturing (i.e., not vacuuming up) larger debris. In an exemplary embodiment, the diameter of the holes may be between 0.1%-40% of the maximum outer diameter of the cup 100. In another exemplary embodiment, the diameter of the holes may be between 1%-25% of the maximum outer diameter of the cup 100. In other exemplary embodiments, the diameter of the holes may be 1%-5%, 5%-10%, 10%-15%, 15%-20%, or 20%-25% of the maximum outer diameter of the cup 100.

The smart evacuation cup 100 may be both sterilizable and reusable, or may be disposable. The material of the smart aerosol evacuation cup 100 may be PLA, ABS, Polypropylene (PP), polysulfone, Ultem®, Polystyrene, Polycarbonate, acetal (POM), PEEK, PEI (ULTEM), FEP, PPSU, PPS, Nylon, paper, and other materials. The cup 100 can be designed and manufactured to be autoclavable and UV sterilizable or can be one-time usage.

The EVAC 100 can also be used to absorb dust and as a spittoon, making it a multifunctional device for dental procedures.

It is to be understood that the above description is intended to be illustrative, and not restrictive. For example, the above described embodiments (and/or aspects thereof) may be used in combination with each other. In addition, many modifications may be made to adapt a particular situation or material to the teachings of the subject matter without departing from its scope. While the dimensions and types of materials described herein are intended to define the parameters of the subject matter, they are by no means limiting and are exemplary embodiments. Many other embodiments will be apparent to one of ordinary skill in the art upon reviewing the above description. The scope of the subject matter described herein should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

This written description uses examples to disclose several embodiments of the subject matter, including the best mode, and also to enable any person of ordinary skill in the art to practice the embodiments disclosed herein, including making and using any devices or systems and performing any incorporated methods. The patentable scope of the subject matter is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

The foregoing description of certain embodiments of the disclosed subject matter will be better understood when read in conjunction with the appended drawings. The various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present subject matter are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described systems and methods, without departing from the spirit and scope of the subject matter herein involved, it is intended that all of the subject matter of the above description or shown in the accompanying drawings shall be interpreted merely as examples illustrating the concepts herein and shall not be construed as limiting the disclosed subject matter.

The flowchart and block diagrams in the figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Words such as “then,” “next,” etc. are not intended to limit the order of the steps; these words are simply used to guide the reader through the description of the methods. Although process flow diagrams may describe the operations as a sequential process, many of the operations can be performed in parallel or concurrently. In addition, the order of the operations may be re-arranged. A process may correspond to a method, a function, a procedure, a subroutine, a subprogram, etc. When a process corresponds to a function, its termination may correspond to a return of the function to the calling function or the main function.

Features or functionality described with respect to certain example embodiments may be combined and sub-combined in and/or with various other example embodiments. Also, different aspects and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, can be at least partially performed via at least one entity or actor in any manner.

Although preferred embodiments have been depicted and described in detail herein, skilled artisans know that various modifications, additions, substitutions, and the like can be made without departing from spirit of this disclosure. As such, these are considered to be within the scope of the disclosure, as defined in the following claims.

What is claimed is:

1. A device for capturing aerosols and debris during oral procedures, comprising:
a hollow main body,
wherein the main body has a first opening at a first end of the main body and a second opening at a second end of the main body;
a rim located at the first end of the main body and surrounding the first opening; and
a coupler portion located at the second end of the main body.

2. The device of claim 1, further comprising:
a cover, sized to occlude the first opening,
wherein the cover includes a plurality of holes.

3. The device of claim 1, wherein the rim has a rim height which is not uniform around the entire first opening.

4. The device of claim 3, wherein the rim height is constant for one half of the first opening and variable for the other half of the first opening.

5. The device of claim 4, wherein the variable rim height is larger than the constant rim height.

6. The device of claim 3, wherein the rim height is constant for a first portion of the first opening and variable for a second portion of the first opening.

7. The device of claim 2, wherein the plurality of holes are all of equal size and are equally distributed over the cover.

8. The device of claim 2, wherein the plurality of holes are distributed more towards the side of the main body with the higher rim height.

9. The device of claim 1, wherein the coupler portion is sized to snuggly fit into or around a vacuum attachment.

10. The device of claim 9, wherein the plurality of holes in the cover are sized to increase a vacuum pressure through the main body.

11. The device of claim 9, wherein the vacuum attachment is part of a high-volume evacuator (HVE).

12. The device of claim 9, wherein the plurality of holes concentrate a vacuum pressure within the main body to each of the plurality of holes.

13. The device of claim 1, wherein the coupler portion is elongated and includes a curve to facilitate placement of the device near a patient's head.

14. The device of claim 1, wherein the main body is funnel shaped.

15. A method, comprising:
connecting the device described in claim 1 to an external vacuum source;
placing the device near a patient's mouth during a dental procedure;
modifying the suction strength to achieve expected results.
